(19) United States
(12) Patent Application Publication
(10) Pub. No.: US 2023/0191009 A1
(43) Pub. Date: Jun. 22, 2023

(54) METHOD OF MANUFACTURING A PUMP, FLUID PUMP, AND DIALYSIS MACHINE

(71) Applicant: B. Braun Avitum AG, Melsungen (DE)

(72) Inventor: Tobias Wuerschmidt, Hann (DE)

(21) Appl. No.: 18/082,324

(22) Filed: Dec. 15, 2022

(30) Foreign Application Priority Data
Dec. 16, 2021 (DE) 10 2021 133 396.2

(51) Int. Cl. A61M 1/16; B23P 15/14

(52) U.S. Cl. CPC A61M 1/1633; B23P 15/14; A61M 2207/10

(57) ABSTRACT

A method of manufacturing pumps of different performances for use in a blood treatment device, preferably a dialysis machine, having a pump housing or a pump housing portion for supportingly holding two gear wheels in meshing engagement. The method includes the step of primary shaping of a universal pump housing blank with all features common to the different pumps as well as with such oversizes as to allow machining for manufacturing all different pumps from the same primary-shaped pump housing blank. The method also includes the step of individualizing the universal pump housing blank by machining in the region of the oversizes to achieve the respective performance.

### CROSS-REFERENCE TO RELATED APPLICATIONS

This application claims priority under 35 U.S.C. § 119 to German Application No. 10 2021 133 396.2, filed Dec. 16, 2021, the content of which is incorporated in its entirety by reference herein.

### FIELD

The present disclosure relates to a method of manufacturing a pump, to a pump for use in a dialysis machine, and to a dialysis machine for extracorporeal treatment of blood.

### BACKGROUND

Gear wheel pumps are widely used flux pumps and have been in use for a long time. In a known gear wheel pump, a gear wheel is driven on a shaft. The driving gear wheel drives a second gear wheel. Both gear wheels run in cylindrical gear wheel holders. There is a respective small gap between a wall of the gear wheel holder and the gear wheels. A liquid is conveyed by the movement of the gear wheels. A gear wheel diameter and/or a gear wheel width are decisive for a delivery rate and a delivery pressure of the gear wheel pump. I.e. the delivery rate and/or the delivery pressure are set by varying the gear wheel diameter, the gear wheel width or both.

It is known to use gear wheel pumps in medical devices, such as dialysis machines. In known dialysis machines, three different types of gear wheel pumps are used in a dialysis fluid circuit. A degassing pump is used to degas the dialysis fluid entering the dialysis machine. A dialysis fluid inlet pump and a dialysis fluid outlet pump are used to balance the dialysis treatment. The different gear wheel pumps are designed for different flow ranges. Thus, the degassing pump has to deliver a higher delivery volume or volume flow rate than the dialysis fluid inlet pump and the dialysis fluid outlet pump, respectively. That is, the degassing pump has to provide a higher performance than the dialysis fluid pumps. Therefore, in known dialysis machines, the degassing pump has thicker gear wheels than the dialysis fluid inlet and outlet pumps. The gear wheel diameters are identical for all three gear wheel pumps. A check valve is installed on the dialysis fluid inlet pump, which opens in the event of overpressure and protects the dialysis machine from excessive pressure.

Due to the higher gear wheel thickness of the degassing pump, a receiving pocket for the gear wheels of this pump has to be deeper than for the other gear wheel pumps. Since all three gear wheel pumps differ, each of the gear wheel pumps has its own pump housing portion in which the gear wheels are supported. Traditionally, the three different pump housing portions are milled or respectively machined from a raw round steel. Machining a different pump housing portion for each of the variations of the gear wheel pumps has the following disadvantages:
new, expensive round materials, such as round steel, are needed for each pump
complex machining
grommet assembly required
a lot of waste
no optimal use of resources.

The milled pump housing portions do not have hose grommets, which are used to connect hoses. These have to be mounted in a separate assembly step. This entails a high level of manual assembly work. It is also possible not to manufacture individual pump housing portions, but to adjust the different gear wheel thicknesses with spacer rings or sealing plates of different thicknesses. This means that the pump housing remains the same and the different gear wheel thicknesses are compensated by spacer rings as placeholders. However, this requires a high degree of manual assembly. It is therefore well known that different pump variants represent a cost factor.

DE 10 2009 047 619 A1 describes a gear wheel pump with a housing ring in which the gear wheels are arranged. The housing ring can be used for different pump versions with different delivery rates and gear wheel thicknesses. A thinner housing ring can be used for the pump version with a lower delivery rate, which has no openings and channels and is therefore cheaper to manufacture. However, the individual housing rings for the respective pump version are manufactured individually by a machining production process.

CN 1 06 870 360 A discloses a housing for a gear wheel pump which consists of a metal-plastic composite and is manufactured by injection molding. Varying the geometry of the housing is associated with high tool and attachment changing costs.

### SUMMARY

The object of the disclosure, therefore, is to overcome or at least reduce the disadvantages of the prior art and preferably to provide a universal pump housing blank or universal pump housing portion blank that can be further processed into a pump housing or pump housing portion for pumps of different performances with minimal effort and at a low cost.

The present disclosure relates to a method of manufacturing pumps of different performances for use in a blood treatment device, preferably a dialysis machine. The pump has a pump housing or a pump housing portion for supportingly holding a fluid delivery device, preferably two gear wheels in meshing engagement. The method according to the disclosure comprises the following steps: primary shaping or molding of a universal pump housing blank or of a universal pump housing portion blank with all the housing features common to the different pumps as well as with such oversizes as to allow machining for manufacturing all the different pumps from the same primary-molded universal pump housing blank or universal pump housing portion blank; and individualizing the universal pump housing blank or universal pump housing portion blank by machining in the region of the oversizes to achieve the respective performance.

The housing features are, for example, a depth of a receiving pocket for the fluid delivery device or gear wheels, a diameter of the receiving pocket, a design of gear wheel receiving surfaces, hose grommets, and the sealing surface. The housing features are preferably machined during the manufacture of the pump housing or pump housing portion from the universal pump housing blank or universal pump housing portion blank.

Machining should be understood as subtractive manufacturing in this context. An example of machining could be milling, drilling or grinding.

In this context, the oversize of the universal pump housing blank or universal pump housing portion blank means that the universal pump housing blank or universal pump housing portion blank is larger than the pump housing or pump housing portion. The oversize is removed by machining during the manufacture of the pump housing portion from the universal pump housing portion blank. For example, the depth of the tooth pockets is 9.5 mm or 10.5 mm, respectively. In the case of the universal pump housing portion blank, the depth of the tooth pockets is less than 9.5 mm. This ensures that sufficient material is available to produce the smallest depth by machining, for example milling. Likewise, the receiving pocket of the pump housing or pump housing portion has a diameter of 12.7 mm. This diameter is smaller for the universal pump housing blank or universal pump housing portion blank and is reworked. The wall thickness of the universal pump housing blank or universal pump housing portion blank is approx. 2.5 mm, which is greater than the wall thickness of the pump housing or pump housing portion. Furthermore, the hose grommets have an inner diameter of approx. 5 mm. The universal pump housing blank or universal pump housing portion blank might has 2 degree mold release slopes.

In other words, the universal pump housing blank or universal pump housing portion blank is manufactured by primary shaping. The universal pump housing blank or universal pump housing portion blank has all the features that are common to the different pumps. Furthermore, the universal pump housing blank or universal pump housing portion blank has such an oversize that all different pumps can be machined from the universal pump housing blank. A machining production technique may include, but is not limited to, milling. For the purposes of the disclosure, however, any other machining operation may also be carried out. In a further step, the universal pump housing blank or universal pump housing portion blank is individualized. In particular, the pump housing or the pump housing portion is machined to such a dimension that the pump can accommodate the suitable fluid delivery device that provides the required performance of the pump.

The universal pump housing blank or universal pump housing portion blank is manufactured by primary molding or shaping. Die casting and metal powder injection molding are stated as possible examples of manufacturing techniques but the list is not exhaustive. However, manufacturing using other primary shaping techniques is also possible.

It should be noted that the individualizing step does not include such machining steps as are carried out as standard in the case of primary molded and in particular cast parts. Such machining steps include in particular deburring or reworking of sealing surfaces, fits or similar surfaces which cannot be produced in sufficient quality by primary shaping.

The performance of the pumps depends on the dimensioning of the pumps. First, the dimensioning of the respective fluid delivery device has an influence on the performance of the pumps. Depending on this, the dimensioning of the pump housing or of the pump housing portion, respectively, which accommodates the fluid delivery device, also has to be dimensioned accordingly. Therefore, in other words, the present disclosure relates to a method of manufacturing a pump housing of a plurality of differently shaped and/or dimensioned pumps of a blood treatment device, preferably of a dialysis machine. The pump housing portion holds the fluid delivery device, preferably two gear wheels in meshing engagement, in a supporting manner. The method comprises the following steps: primary shaping of a universal pump housing blank or universal pump housing portion blank with all features common to the different pumps as well as with such oversizes that allow machining to produce all different pumps from the same primary-molded pump housing blank. Individualizing the universal pump housing blank or universal pump housing portion blank by machining to adapt it to the individual construction features and/or dimensioning of the respective pump to achieve different performance specifications.

If the pump is a gear wheel pump, the fluid delivery device comprises two gear wheels that mesh with each other. The pump housing portion is then a gear wheel holder in which the gear wheels are rotatably mounted. By varying a gear wheel thickness, the performance of the gear wheel pump can be adjusted. In the present case, a gear wheel holder that accommodates the gear wheels is milled to such a depth that it can accommodate exactly the gear wheel thickness required for the respective pump performance.

The method according to the disclosure for manufacturing the pumps with different performance has the following advantages. The universal pump housing blank or universal pump housing portion blank can be further processed into the pump housing or pump housing portion for the pumps of different performance in just a few machining steps. In particular, the dimensioning of the receiving pocket for gear wheels of different thicknesses is possible with only a few machining steps. This in turn makes it possible to manufacture pumps of different performance at low cost. Since the universal pump housing blank or universal pump housing portion blank can be used for all pump variations, it can be produced in large quantities. This reduces the manufacturing costs per piece. All other components of the pump are the same and can thus be provided in large quantities. Furthermore, the pump housing or the pump housing portion is not milled, in contrast to conventional production. Production by primary shaping results in significantly less waste and material can be saved. Thus, in particular, material consumption/rejects can be reduced by primary shaping or molding manufacturing as compared to machining manufacturing. By primary shaping, thinner wall thicknesses of the pump housing portion can be achieved, which further reduces material consumption. In addition, out-of-tool universal pump housing blanks can be produced without the need for costly post-processing. This saves assembly steps and thus costs. In particular, the adaptation of the pump housing portion to the different gear wheel thicknesses via sealing plates of different thicknesses is eliminated compared with the conventional manufacturing process. The manufacturing method according to the disclosure can therefore be used to manufacture different pumps, in particular a degassing pump, a dialysis fluid inlet pump and a dialysis fluid outlet pump, at low costs.

To summarize, the core of the disclosure is that a general universal pump housing blank or universal pump housing portion blank for a pump is produced by primary molding or shaping, which is further processed into different pump housing portions sized precisely to accommodate fluid delivery units that provide the performance required by the pump.

According to an optional feature of the disclosure, during individualizing the universal pump housing blank, receiving pockets for the fluid delivery device are configured, in particular enlarged or reduced by machining, in such a way that the fluid delivery device can be accommodated that has corresponding dimensions and provides the respectively required performance.

As described above, the performance of the pump depends on the dimensioning of the fluid delivery device. The dimensioning of the pump housing portion has to be adapted to the respective fluid delivery device. Therefore, when individualizing the universal pump housing blank or the universal pump housing portion blank, receiving pockets are enlarged or reduced in size in such a way that the receiving pockets accommodate exactly the fluid delivery device that is adapted to the required performance. The receiving pockets are enlarged or reduced in size by machining, in particular milling. In this way, pumps with different performance can be manufactured by simply adapting the dimensions of the receiving pocket of the pump housing portion. All other components of the pump and the dimensions of the pump housing portion are the same for pumps with different performance.

According to a further optional feature of the disclosure, during individualizing the universal pump housing blank or universal pump housing portion blank, gear wheel support surfaces of the receiving pockets are machined axially in order to increase a depth of the receiving pockets. For pumps where high performance is required, the gear wheels have to be correspondingly thick. For the thick gear wheels, the receiving pockets have to be correspondingly large. In order for the receiving pockets to be able to accommodate the gear wheels, the gear wheel support surfaces are milled in such a way that the receiving pockets are deeper and can also accommodate thick gear wheels. By milling the gear wheel support surfaces, the universal pump housing blank or universal pump housing portion blank can be processed with little effort into a pump housing or pump housing portion for a pump with high performance. Alternatively or in addition to this, the diameters of the receiving pockets may also be enlarged by machining so that, for example, gear wheels with larger diameters can be used.

The size of the receiving pockets is defined by the distance from a connecting flange of the pump housing or pump housing portion to the gear wheel support surfaces. The connecting flange defines the surface over which the gear wheels must not protrude. The gear wheel support surface is the surface on which the gear wheels rotate in the receiving pockets.

According to a further optional feature of the disclosure, an axial extension of the universal pump housing blank or universal pump housing portion blanks has an oversize. That is, the universal pump housing blank or universal pump housing portion blank has a longer axial extension than the pump housing or pump housing portion and is shortened by axial machining to reduce the size of the receiving pocket.

In other words, the universal pump housing blank or universal pump housing portion blank has more material than each of the pump housing portions into which the universal pump housing blank or universal pump housing portion blank is further processed. Therefore, each of the different pump housing portions can be manufactured from the universal pump housing blank by machining. The axial extension of the universal pump housing blank or universal pump housing portion blank also includes a connecting flange formed on one side of the pump housing portion. The connecting flange can be milled to reduce the receiving pocket for the gear wheels. The distance between the gear wheel support surface and the connecting flange limits the thickness of the gear wheels that can be accommodated between these surfaces. If the connecting flange is milled off, this distance is reduced.

The connecting flange is defined as the front side of the universal pump housing blank or universal pump housing portion blank that faces in the direction of the gear wheels and includes both a surface for receiving a sealing plate and a surface for connecting to a drive housing of the pump. The axial extension is to be understood as an extension in the direction of a drive shaft of the pump.

According to a further optional feature of the disclosure, during individualizing the universal pump housing blank or universal pump housing portion blank, the inner sides of the receiving pockets are machined off in order to increase the diameters of the receiving pockets. It is possible to mill off the gear wheel support surface to increase the depth of the receiving pocket and to achieve higher pump performance through thicker gear wheels. It is also possible to radially mill the inner surfaces of the receiving pockets to increase the diameter of the receiving pockets. This allows gear wheels with a larger diameter to be used in the pump and the larger gear wheel diameter increases the performance of the pump.

According to a further optional feature of the disclosure, when the universal pump housing blank is primary shaped, hose grommets are formed that are integrally formed with the universal pump housing blank. The hose grommets are configured and prepared to connect fluid carrying hoses thereto. The hose grommets may be primary shaped as an integral part of the universal pump housing blank. With conventional machining of the individual pump housing portions, the hose grommets have to be manually assembled afterwards. This assembly is eliminated by the method according to the disclosure. This saves working time during assembly and thus costs.

According to a further optional feature of the disclosure, when individualizing the universal pump housing blank, a (thoroughfare) channel is drilled out from gear wheel support surfaces of the receiving pocket to a connection nozzle for attaching a check valve. The dialysis fluid inlet pump has a check valve that opens in the event of overpressure and protects the dialysis machine from excessive pressure. If the pressure in the dialysis machine is too high, pressure can be released via the check valve. The check valve is attached to mounting spigots that protrude from the universal pump housing blank. The check valve is connected to connection nozzles of the universal pump housing blank. In order to establish a fluid connection between the inside of the pump and the check valve, the thoroughfare channel of the gear wheel support surfaces of the receiving pocket and the connection nozzles is drilled out. The connection nozzles for the check valve are configured in the universal pump housing blank. For pumps other than the dialysis fluid inlet pump, the thoroughfare channel is not drilled open and thus no fluid connection is established between the receiving pocket or the gear wheel chamber and the exterior.

By drilling open of the thoroughfare channel, a further pump variation, i.e. the dialysis fluid inlet pump, can be produced at low costs. In order to produce a pump housing portion for the dialysis fluid inlet pump from the universal pump housing blank, the gear wheel support surfaces of the receiving pocket are milled and the thoroughfare channel is drilled open.

According to a further optional feature of the disclosure, the connecting flange and a mounting flange of the drive housing are connected to each other exclusively by interposing seals without additional spacer rings for individual adjustment of the axial dimension of the receiving pockets. The connecting flange and the mounting flange are screwed together. The connecting flange has a thread for this purpose. A separating can is also screwed between the two flanges to seal the gear wheels axially. Furthermore, a sealing plate is attached to the connecting flange, which also seals the gear wheels axially from the magnetic coupling. These seals are the only components located between the connecting flange and the mounting flange. Conventionally, the different gear wheel thicknesses of different pumps are compensated for by sealing plates or spacer rings of different thicknesses. Since the receiving pockets of the pump housing or pump housing portion of the present disclosure compensate for the different gear wheel thicknesses, the use of additional spacer rings or sealing plates of different thicknesses is not necessary.

The object of the present disclosure is further solved by a pump, preferably a gear wheel pump, for use in a blood treatment device, preferably a dialysis machine. The pump comprises an electric drive, in particular an electric motor, with a rotor mounted or formed on a drive shaft and supported in a drive housing having a mounting flange. Furthermore, the pump has a pump housing or pump housing portion with a connecting flange. The pump housing or pump housing portion is manufactured according to the disclosed method by individualizing the universal pump housing blank or universal pump housing portion blank and is flanged to the mounting flange via the connecting flange. The connecting flange is in turn attached to the mounting flange in such a way that the drive shaft projects into an interior space of the pump housing or pump housing portion formed jointly by the receiving pockets. Furthermore, the pump has the fluid delivery device in the form of two gear wheels in meshing engagement, which are in operative engagement with the drive shaft and slide against the inner side of the receiving pockets.

In other words, the electric motor drives the gear wheels of the pump directly or possibly via appropriate clutches or a gearbox. The gear wheels are received in the pump housing or pump housing portion in the receiving pocket. The pump housing or pump housing portion is manufactured from the universal pump housing blank or universal pump housing portion blank in accordance with the disclosed method. The receiving pocket is dimensioned in such a way that the gear wheels fit into the receiving pockets.

The method according to the disclosure includes primary molding or shaping of the universal pump housing blank and subsequent individualization of the primary-molded universal pump housing blank to form different pump housing portions. Since the individualization requires only a few production steps, assembly and machining costs can be saved. As a result, pumps of different performance can be manufactured cost-effectively from just one universal pump housing blank. The various pump housing portions of the respective pumps can be manufactured from the universal pump housing blank in a small number of manufacturing steps. In particular, material consumption is reduced as compared with machining. The fact that only a few manufacturing steps are required for the (machining) finishing of the cast part or respectively of the cast universal pump housing blank means that working time and thus costs can be saved. The various pumps differ from each other in terms of performance and other aspects, such as the presence of a check valve. As explained above, the different pump housing portions are made from the same universal pump housing blank. The remaining components of the different pumps are all the same and can therefore also be manufactured inexpensively in large quantities. Thus, the different pumps can be manufactured inexpensively from the same components.

According to a further optional feature of the disclosure, the connecting flange and the mounting flange are connected to each other exclusively by interposing seals without additional spacer rings for individual adjustment of the axial dimension of the receiving pockets. The connecting flange and the mounting flange are screwed together such that the separating can is screwed between the two flanges. The gear wheels of the pump are axially sealed by the sealing plate and the separating can. When connecting the connecting flange and the mounting flange, no additional sealing plates or spacer rings are required, whose axial dimensions compensate for the axial dimensions of the gear wheels. According to the disclosure, the different gear wheel thicknesses are compensated for by the dimensions of the pump housing or pump housing section.

According to a further optional feature of the disclosure, the gear wheels are mounted on cylindrical pins that are pressed into locating bores of the pump housing or pump housing portion. Thus, no additional bearings have to be installed.

According to a further optional feature of the disclosure, the gear wheels are mounted on slide bearings in the pump housing portion. The use of the slide bearings reduces friction between the gear wheels and the pump housing portion.

According to a further optional feature of the disclosure, the gear wheels are mounted on sliding solid shafts in the pump housing portion. Thus, no additional bearings have to be installed for the gear wheels. This saves an additional manufacturing step and thus costs and labor.

According to a further optional feature of the disclosure, the pump housing portion has a wall thickness suitable for casting. The wall thickness of the pump housing portion is less than that of a conventionally machined pump housing portion. On the one hand, this is advantageous since it prevents blowholes from forming during casting or primary shaping, respectively. On the other hand, the low wall thickness improves the thermal behavior of the gear wheel pump. Since the gear wheels are made of a plastic, they have a different coefficient of thermal expansion than the metal pump housing portion. When heated, the gear wheels expand faster than the pump housing portion and may therefore jam. This risk is reduced by the thinnest possible wall thickness of the pump housing portion, since the smaller mass heats up faster and thus expands more quickly. This can prevent the gear wheels, which are made of a plastic such as PEEK, from expanding faster than the pump housing portion due to their higher coefficients of thermal expansion and thus jamming. As a result, the tolerance between the inside of the pump housing portion and the respective gear wheel can be tightened, leading to better pumping performance or delivery rate of the gear wheel pump.

In this context, wall thickness suitable for casting is to be understood as a wall without material accumulations. The wall also does not exhibit abrupt changes in wall thickness. The lack of material accumulations reduces the wall thickness. This also reduces possible thermal distortion of the wall of the pump housing portion.

It is also conceivable that the pump housing portion is also made of a plastic, for example PEEK (polyetheretherketone) or POM (polyoxymethylene).

The present disclosure further relates to a dialysis machine for extracorporeal purification of blood comprising a plurality of pumps of different performance, preferably gear wheel pumps, manufactured according to the method according to the disclosure. The dialysis machine comprises a plurality of gear wheel pumps which are provided and configured for degassing and pumping fluids. This involves a degassing pump for degassing a fluid, in particular dialysis fluid. The gear wheel pumps are further a dialysis fluid inlet pump and a dialysis fluid outlet pump for balancing the dialysis fluid.

The different gear wheel pumps have different functions and different requirements. For example, the required flow rate/flow quantity/volume flow of the gear wheel pumps differs. The degassing pump has to deliver a higher volume flow than the other gear wheel pumps and therefore has a larger gear wheel thickness. The dialysis fluid inlet pump has a check valve to protect the dialysis machine from excessive pressure. Due to these differences, the respective gear wheel pumps have differently designed/constructed pump housing portions. For example, the pump housing portion of the degassing pump is deeper than the pump housing portions of the other pumps to accommodate the thicker gear wheels of the degassing pump. These differently designed pump housing portions are nevertheless made from the same/the identical universal pump housing blank. The universal pump housing blank is manufactured by out-of-tool primary shaping and has to be machined only minimally after primary shaping. The receiving pocket of each pump is dimensioned in such a way that the gear wheels that deliver the required performance can be accommodated or, respectively, fit into the receiving pocket.

In summary, the present disclosure has the following advantages:
inexpensive to manufacture;
modular construction system;
efficient use of materials;
no assembly of hose grommets necessary;
lower tendency of gear wheel jamming during temperature changes due to lower wall thicknesses;
lower weight; and
space saving.

### BRIEF DESCRIPTION OF THE DRAWINGS

FIG. 1 shows a pump according to the disclosure according to a first embodiment;

FIG. 2 shows a longitudinal section through the pump according to the disclosure according to FIG. 1;

FIG. 3a shows an isometric view of a universal pump housing blank;

FIG. 3b shows a sectional view of the universal pump housing blank in FIG. 3a;

FIG. 3c shows a sectional view of a pump housing portion for a degassing pump according to the first embodiment;

FIG. 3d shows a sectional view of a pump housing portion for a dialysis fluid inlet pump according to a second embodiment;

FIG. 3e shows a sectional view of a pump housing portion for a dialysis fluid outlet pump according to a third embodiment;

FIG. 4 shows a sectional view through a pump according to the disclosure according to a fourth embodiment;

FIG. 5 shows a schematic view of a dialysis machine according to the disclosure; and

FIG. 6 shows a schematic view of two gear wheels in meshing engagement.

### DETAILED DESCRIPTION

Embodiments of the present disclosure are described below based on the accompanying figures.

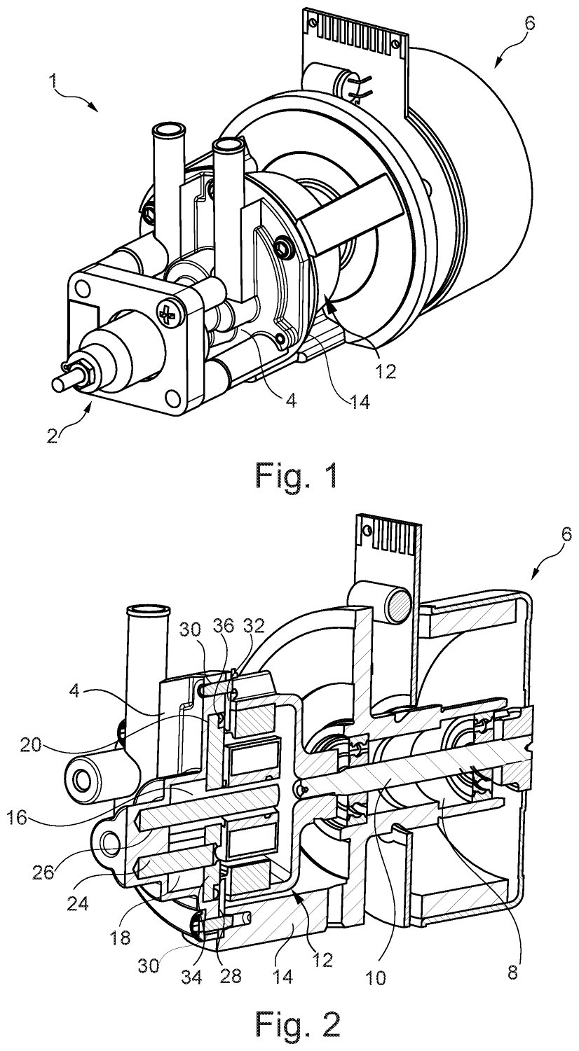

FIG. 1 shows a pump 1 according to a first embodiment. The pump 1 according to the first embodiment is in particular a dialysis fluid inlet pump for a blood treatment device, preferably a dialysis machine. The dialysis fluid inlet pump differs from other pumps 1 by a check valve 2, which is screwed to a pump housing or pump housing portion 4 of the pump 1. FIG. 2 shows a longitudinal section through a pump 1 according to the disclosure without the check valve 2. The pump 1 has an electric drive 6, preferably an electric motor. The electric drive 6 has a rotor 8 mounted on a drive shaft 10. The electric drive 6 is connected to a magnetic coupling 12 via the drive shaft 10. The magnetic coupling 12 is mounted within a drive housing 14. A magnetic force-fit connection of the magnetic coupling 12 brings the drive shaft 10 into operative engagement with a first gear wheel 16. The first gear wheel 16 meshes with and drives a second gear wheel 18. The two gear wheels 16, 18 are rotatably mounted in the pump housing or the pump housing portion 4. The pump housing portion 4 has a substantially round base plate 19 with a connecting flange 20. For receiving the gear wheels 16, 18, the pump housing portion 4 forms an (oval) receiving pocket 22 in the base plate 19. The two gear wheels 16, 18 are each rotatably mounted on a cylindrical pin 24, which are pressed into locating bores 26 of the pump housing or pump housing portion 4. The pump housing portion 4 is described in detail below. The connecting flange 20 of the pump housing portion 4 is threaded to a mounting flange 28 of the drive housing 14. For this purpose, the connecting flange 20 has threads 30 prepared to receive screws with which the connecting flange 20 and the mounting flange 28 are screwed together. The pump housing portion 4 and the drive housing 14 are sealed with a separating can 32. The separating can 32 is screwed through the threads 30 between the pump housing portion 4 and the drive housing 14. The two gear wheels 16, 18 are sealed from the magnetic coupling 12 and the interior of the drive housing 14 with a sealing plate 34. The pump 1 is sealed to the outside with a sealing ring 36.

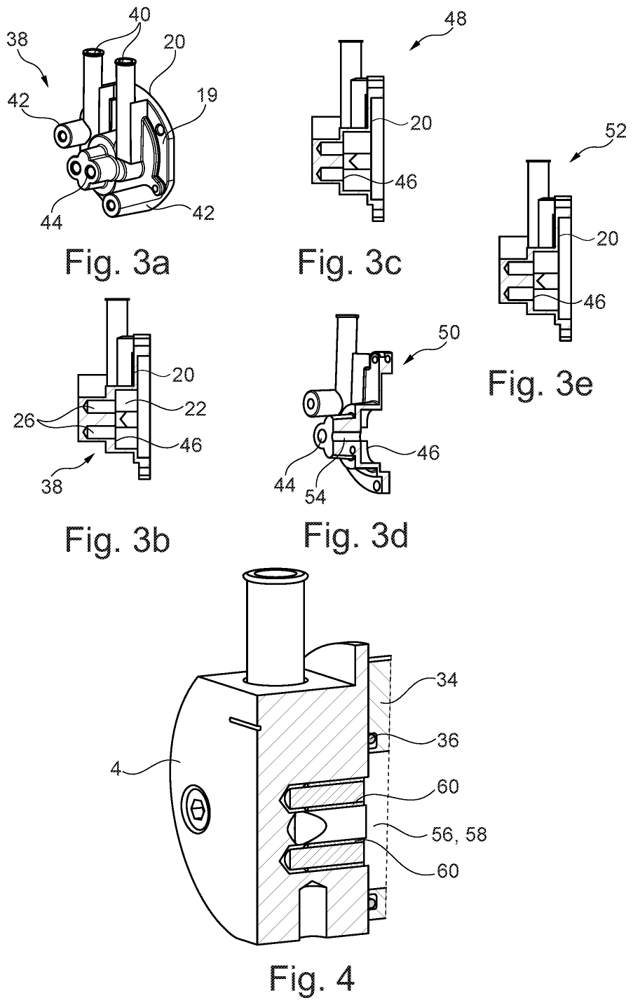

FIG. 3a shows a universal pump housing blank or universal pump housing portion blank 38 before further processing or machining. The universal pump housing blank 38 has the substantially round base plate 19 with the connecting flange 20 on one side of the base plate 19. Various connections are configured on the opposite side of the connecting flange 20. The universal pump housing blank 38 has two hose grommets 40 for connecting hoses (not shown). Furthermore, the universal pump housing blank 38 has two mounting spigots 42 for screwing on or fixing the check valve 2 and two connection nozzles 44 for connecting the check valve 2. In the universal pump housing blank 38 before further processing, the connection nozzles 44 are not drilled through or open, or respectively are not continuous, i.e. there is no fluid connection between the connection nozzles 44 and the interior of the receiving pocket 22.

FIG. 3b shows a sectional view of the universal pump housing blank 38 in FIG. 3a. The universal pump housing blank 38 has the two locating bores 26 in which the gear wheels are mounted via the cylindrical pins (not shown in FIG. 3b). The universal pump housing blank 38 furthermore has the receiving pocket 22 for the two gear wheels 16, 18 (not shown in FIG. 3b). The gear wheels 16, 18 are seated on a gear wheel contact surface 46, i.e. the gear wheels 16, 18 slide along the gear wheel contact surface 46 during rotation. The universal pump housing blank 38 is prepared to be converted by machining into one of three variants of the pump housing or pump housing portion 4.

The three variants are a pump housing portion for a degassing pump 48, a pump housing portion for a dialysis fluid inlet pump 50 and a pump housing portion for a dialysis fluid outlet pump 52. The pump housing portion for the degassing pump 48 shown in FIG. 3c and the pump housing portion for the dialysis fluid outlet pump 52 shown in FIG. 3e differ from each other only in the depth of the receiving pocket 22 for the gear wheels 16, 18. A degassing pump is designed for higher performance than the other pumps 1 and therefore requires gear wheels 16, 18 with a higher gear wheel thickness. For the thicker gear wheels 16, 18, the pump housing portion for the degassing pump 48 has a deeper receiving pocket 22 than the pump housing portion for the dialysis fluid outlet pump 52. The pump housing portion for the dialysis fluid inlet pump 50 is shown in FIG. 3d and has the same dimensions as the pump housing portion for the dialysis fluid outlet pump 52. I.e. the receiving pocket 22 of the pump housing portion for the dialysis fluid inlet pump 50 is as deep as the receiving pocket 21 of the pump housing portion for the dialysis fluid outlet pump 52. One difference in the pump housing portion for the dialysis fluid inlet pump 50 is that one or more thoroughfare channels 54 is or are bored open from the gear wheel contact surface 46 to the connection nozzles 44. Through the thoroughfare channels 54, the gear wheel contact surface 46 is in fluidic contact with the check valve 2 of the dialysis fluid inlet pump.

FIG. 4 shows a section of a cross-section through a pump 1 according to a further embodiment. Two gear wheels 56, 58 are mounted on slide bearings 60 in the pump housing or pump housing portion 4. For this purpose, only the bearing fits and the wall thicknesses have to be adapted. Here, the gear wheels 56, 58 are not mounted on cylindrical pins as in the first embodiment. Rather, the gear wheels 56, 58 have a protruding stub 61. It is further conceivable to mount the gear wheels 56, 58 on sliding solid shafts (not shown) in the pump housing portion 4. In both variants, lubrication of the bearings has to be ensured.

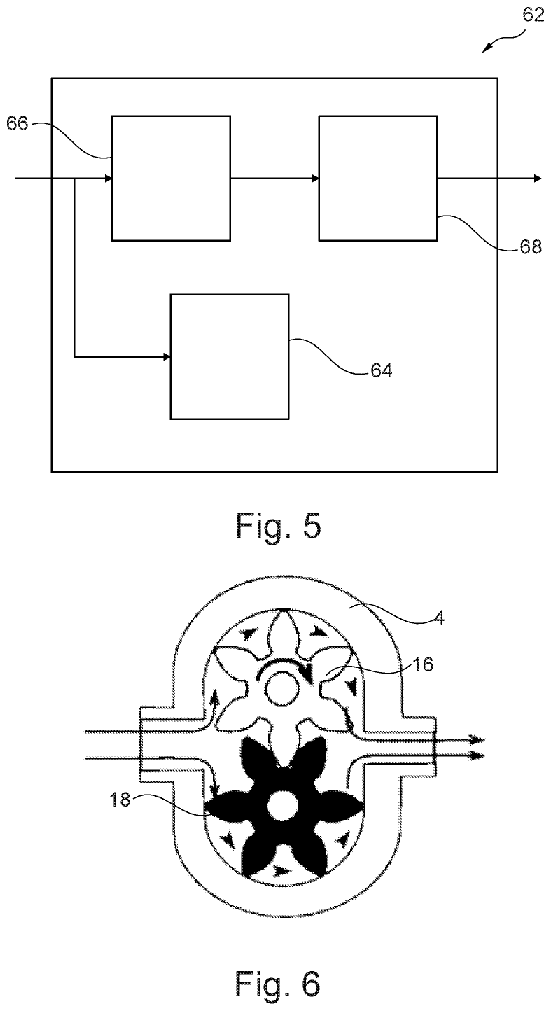

FIG. 5 shows a schematic view of a dialysis machine 62 according to the disclosure. The dialysis machine 62 is a blood treatment device for extracorporeal treatment of blood. The dialysis machine 62 comprises a blood circuit and a dialysis fluid circuit. The dialysis fluid circuit has three different pumps 1. The first pump is a degassing pump 64, the second pump is a dialysis fluid inlet pump 66, and the third pump is a dialysis fluid outlet pump 68. The degassing pump 64 is provided to degas the dialysis fluid. The dialysis fluid inlet pump 66 and the dialysis fluid outlet pump 68 are provided for balancing the dialysis fluid during blood treatment. The different pumps 1 each have different requirements. The degassing pump 64 has to deliver a higher performance than the other pumps. That is, the volume flow rate that the degassing pump 64 has to deliver is greater than that of the other pumps. Therefore, the degassing pump 64 has gear wheels 16, 18 with a higher gear wheel thickness. The dialysis fluid outlet pump 68 is identically constructed to the degassing pump 64 except for the gear wheel thickness. The gear wheels 16, 18 of the dialysis fluid outlet pump 68 are thinner than those of the degassing pump 64. The dialysis fluid inlet pump 66 has the same gear wheel thickness as the dialysis fluid outlet pump 68. However, the dialysis fluid inlet pump 66 has the check valve 2, which opens in the event of overpressure and protects the dialysis machine 62 from excessive pressures. In the dialysis fluid inlet pump 66, one or more thoroughfare channels 54 are bored between the receiving pocket 22 of the dialysis fluid inlet pump 66 and the exterior of the pump. The check valve 2 is placed on this thoroughfare channel 54.

The degassing pump 64 has gear wheels 16, 18 with a width of preferably 10.5 mm. The dialysis fluid outlet pump 68, on the other hand, has gear wheels 16, 18 with a width of preferably 9.5 mm. The additional check valve 2 is attached to the dialysis fluid inlet pump 66, which opens when the pressure is too high and protects the dialysis machine 62 from overload/overpressure. The universal pump housing blank 38 is identical for all pumps 1. The universal pump housing blank 38 only has to be slightly machined for the respective application. For the degassing pump 64, the receiving pocket 22 is milled to a depth of preferably 10.5 mm. For the dialysis fluid outlet pump 68 and the dialysis fluid inlet pump 66, the receiving pocket 22 is milled to a depth of preferably 9.5 mm. For the dialysis fluid inlet pump 66, additional threads 70 are inserted at the mounting spigot 42 and thoroughfare channels 54 to accommodate the check valve 2.

For the degassing pump 64, the gear wheel contact surface 46, bearing fits and sealing surface are milled into the universal pump housing blank 38. Threads 30 are inserted or cut for mounting the separating can 32. The receiving pocket 22 is drilled out to 10.5 mm. The same machining steps are carried out for the dialysis fluid outlet pump 68 and dialysis fluid inlet pump 66. Only the depth of the receiving pocket is machined to 9.5 mm. Thread 70 is inserted into the outer receptacles of dialysis fluid inlet pump 66 to accommodate the check valve 2, and the thoroughfare channels 54 from gear wheels 16, 18 to the check valve 2 are drilled open.

FIG. 6 shows the two gear wheels 16, 18 meshing with each other. The two gear wheels 16, 18 are arranged in the pump housing or pump housing portion 4. The fluid, preferably the dialysis fluid, is conveyed by the rotation of the gear wheels 16, 18.

The object of the disclosure can be solved not only by a gear wheel pump with the gear wheels 16, 18 as fluid delivery device. It is further possible to use a gear ring pump or an internal gear pump. Even with an internal gear pump, the delivery rate of the pump depends on the gear wheel thickness, for example. This means that a universal pump housing blank 38 with variable depth of the receiving pocket for the gear wheels can also be used to vary the output or performance of the internal gear pump.

1. A method of manufacturing pumps, each pump having a unique performance for use in a blood treatment device having a pump housing or a pump housing portion for supportingly holding gear wheels in meshing engagement, the method comprising the steps of:
primary shaping of a universal pump housing blank or a universal pump housing portion blank with housing features common to the pumps and with an oversize to allow machining for manufacturing the pumps from the primary-shaped universal pump housing blank or the universal pump housing portion blank; and
individualizing the universal pump housing blank or the universal pump housing portion blank by machining in a region of the oversize to achieve the unique performance.

2. The method according to claim 1, wherein during individualizing the universal pump housing blank or the universal pump housing portion blank, receiving pockets for the gear wheels are configured to accommodate the gear wheels, wherein the gear wheels provide the unique performance.

3. The method according to claim 2, wherein the receiving pockets are configured by enlarging or reducing through machining.

4. The method according to claim 2, wherein during individualizing the universal pump housing blank or the universal pump housing portion blank, gear wheel support surfaces of the receiving pockets are machined radially to increase a diameter of the receiving pockets.

5. The method according to claim 2, wherein during the step of individualizing the universal pump housing blank or universal pump housing portion blank, axial inner sides of the receiving pockets are axially machined off in order to increase a depth of the receiving pockets.

6. The method according to claim 2, wherein an axial extension of the universal pump housing blank or the universal pump housing portion blank has the oversize and is shortened by axial machining to axially shorten the receiving pockets.

7. The method according to claim 6, wherein a connecting flange is axially oriented with the universal pump housing blank or the universal pump housing portion blank and is connected to a drive by a mounting flange, and
wherein the oversize is configured on the connecting flange of the universal pump housing blank or universal pump housing portion blank and which is axially milled off to reduce the receiving pockets.

8. The method according to claim 7, wherein the connecting flange and the mounting flange are connected to each other exclusively by interposing seals without additional spacer rings.

9. The method according to claim 1, wherein hose grommets are formed during the primary shaping of the universal pump housing blank or the universal pump housing portion blank, the hose grommets being integrally formed with the universal pump housing blank or the universal pump housing portion blank.

10. A method of manufacturing a degassing pump comprising the following steps:
primary shaping of a universal pump housing blank or a universal pump housing portion blank;
milling of a gear wheel contact surface, bearing fits and a sealing surface into the primary shaped universal pump housing blank or the universal pump housing portion blank;
thread cutting of threads for a separating can; and
drilling of a receiving pocket for gear wheels.

11. A method of manufacturing a pump, the pump being a dialysis fluid inlet pump for a dialysis machine, the method comprising the following steps:
primary shaping of a universal pump housing blank or a universal pump housing portion blank for the pump;
milling of a gear wheel contact surface, bearing fits and a sealing surface into the primary shaped universal pump housing blank or the universal pump housing portion blank;
thread cutting of threads for a separating can;
drilling of a receiving pocket for gear wheels; and
boring of channels for a check valve between the receiving pocket and an outer side of the pump.

12. A pump for use in a blood treatment device comprising:
a pump housing or a pump housing portion manufactured according to the method of claim 1, the pump housing or the pump housing portion comprising receiving pockets;
an electric drive with a rotor mounted or formed on a drive shaft and supported in a drive housing having a mounting flange; and
two gear wheels in meshing engagement and in operative engagement with the drive shaft, the two gear wheels sliding against inner sides of the receiving pockets,
the pump housing or the pump housing portion being flanged to the mounting flange, in such a way that the drive shaft projects into an interior space of the pump housing or the pump housing portion.

13. The pump according to claim 12, wherein the connecting flange and the mounting flange are connected to each other exclusively by interposing seals without additional spacer rings for individual adjustment of the axial dimension of the receiving pockets.

14. The pump according to claim 12, wherein the two gear wheels are mounted on cylindrical pins that are pressed in the pump housing or the pump housing portion.

15. The pump according to claim 12, wherein the two gear wheels are mounted in the pump housing or the pump housing portion with slide bearings.

16. The pump according to claim 12, wherein the two gear wheels are mounted on a directly sliding solid shaft in the pump housing or the pump housing portion.

17. The pump according to claim 12, wherein the electric drive is an electric motor.

18. The pump according to claim 12, wherein the two gear wheels comprise external teeth which are in meshing engagement.

19. The pump according to claim 12, wherein the two gear wheels comprise a greater diameter gear wheel with internal teeth and a smaller diameter gear wheel with external teeth, the greater diameter gear wheel surrounding the smaller diameter gear wheel, and the smaller diameter gear wheel being arranged eccentrically in the greater diameter gear wheel.

20. A dialysis machine for extracorporeal purification of blood comprising a plurality of pumps manufactured by the method according to claim 1.
